(19) United States
(12) Patent Application Publication
(10) Pub. No.: US 2014/0296946 A1
(43) Pub. Date: Oct. 2, 2014

(54) LASING HEADGEAR FOR MEDIATING HAIR GROWTH

(71) Applicants: Sean Shahram Malek, Las Vegas, NV (US); Shane Malek, Henderson, NV (US)

(72) Inventors: Sean Shahram Malek, Las Vegas, NV (US); Shane Malek, Henderson, NV (US)

(21) Appl. No.: 14/231,461

(22) Filed: Mar. 31, 2014

Related U.S. Application Data
(60) Provisional application No. 61/807,083, filed on Apr. 1, 2013.

(51) Int. Cl. A61N 5/06

(52) U.S. Cl. CPC A61N 5/0617
USPC 607/89

(57) ABSTRACT

A headgear lasing system, including: a power supply; a laser device coupled in power receiving relationship to the power supply, and configured for generating laser radiation effective to promote growth of head hair; a multiplicity of optical fibers coupled with the laser device to receive the laser radiation for transmission thereof through the optical fibers to respective end portions thereof; and a support that is configured to overlie a head region, and to which the end portions of the optical fibers are secured so that the laser radiation transmitted through the optical fibers is directed therefrom to the head region. The support may be a separate structural component that is secured to an existing headgear article, or the support may be a constituent part of a headgear article, such as a crown portion of a cap or hat article. The headgear lasing system is usefully employed to impinge lasing radiation on a scalp region of a wearer, to mediate hair growth on such scalp region. Also disclosed are headgear lasing systems that additionally comprise scalp massaging capability, for contemporaneous treatment of the scalp region with lasing radiation and massage.

### CROSS-REFERENCE TO RELATED APPLICATION

The benefit under 35 USC 119(e) of U.S. Provisional Patent Application 61/807,083 filed Apr. 1, 2013 in the names of Sean Shahram Malek for “Lasing Headgear for Mediating Hair Growth” is hereby claimed. The disclosure of U.S. Provisional Patent Application 61/807,083 is hereby incorporated herein by reference, in its entirety, for all purposes.

### FIELD

The present disclosure relates to a headgear lasing system for delivery of photoenergy to the scalp for mediating hair growth, and to corresponding method of use of such system, as well as to apparel items comprising such system.

### DESCRIPTION OF THE RELATED ART

The loss of head hair as a result of alopecia and other physiological conditions has given rise to a wide variety of approaches for combating and reversing such loss. These approaches include therapeutic compositions containing minoxidil or other therapeutic agents that are topically applied to the scalp according to a regular dosage regimen, surgical interventions such as implantation of hair plugs and other transplantation techniques, and wigs that are worn on the head.

A particularly common hair loss condition is male pattern baldness, in which hair loss occurs at the crown area of the scalp. The scalp areas exposed as a result of such condition are particularly susceptible to sun exposure, in consequence of which persons having such baldness frequently wear caps, hats, and other headgear to protect the bald areas from sunburn and the other adverse effects, sun exposure.

Accordingly, the art continues to seek improved treatment approaches for combating and reversing loss of head hair.

### SUMMARY

The present disclosure relates to systems for combating and reversing loss of head hair, and more particularly a headgear lasing system, and associated methods and apparel items.

In one aspect, the disclosure relates to a headgear lasing system, comprising:

a power supply;
a laser device coupled in power receiving relationship to the power supply, and configured for generating laser radiation effective to promote growth of head hair;
a multiplicity of optical fibers coupled with the laser device to receive said laser radiation for transmission thereof through said optical fibers to respective end portions thereof; and
a support that is configured to overlie a head region, and to which the end portions of the optical fibers are secured so that said laser radiation transmitted through said optical fibers is directed therefrom to the head region.

In another aspect, the disclosure relates to a method of enhancing growth of head hair of a subject in need thereof, comprising generating laser radiation effective for promoting growth of head hair, transmitting said radiation through a multiplicity of optical fibers, and impinging said radiation from distal end portions of the optical fibers on the head of said subject.

In a further aspect, the disclosure relates to a method of enhancing growth of head hair of a subject in need thereof, comprising disposing on the head of said subject a headgear lasing system according to the present disclosure, and operating same to expose the head of said subject to said laser radiation.

In another aspect, the disclosure relates to a scalp conditioning system including a laser source configured for generating laser radiation effective to promote growth of head hair, a vibration energy source configured for generating vibration energy, and an array of bristle members arranged to overlie a scalp region so that terminal end portions of the bristle members are in contact with the scalp, such bristle members being optically coupled to the laser source and adapted to transmit lasing energy from the laser source to the scalp region when the laser source is energized, and such bristle members being coupled to the vibration energy source so that the terminal end portions of the bristle members provide vibratory massaging action on the scalp region, when the vibration energy source is energized.

A still further aspect of the disclosure relates to a headgear article comprising a scalp conditioning system as above described.

Yet another aspect of the disclosure relates to a scalp conditioning method in which lasing energy is impinged on a scalp region and vibratory massaging energy is transmitted to the scalp region, contemporaneously, sequentially, or otherwise selectively, from a scalp conditioning system of the disclosure.

Other aspects, features and embodiments of the disclosure will be more fully apparent from the ensuing description and appended claims.

### BRIEF DESCRIPTION OF THE DRAWINGS

FIG. 1 is a schematic representation of a headgear lasing system according to one embodiment of the present disclosure.

FIG. 2 is a schematic representation of a cap incorporating the headgear lasing system of FIG. 1.

FIG. 3 is a schematic representation of a headgear article according to another embodiment of the disclosure, providing capability of impinging lasing energy on the scalp of a wearer and/or massaging of the scalp of the wearer.

FIG. 4 is a perspective view of a portion of a bristle member of the headgear article of FIG. 3, incorporating an optical fiber extending through a hollow bristle element.

FIG. 5 is a perspective view of a remote control unit that may be utilized with the headgear article shown in FIG. 3.

FIG. 6 is a rear perspective view of the headgear article of FIG. 3, showing the adjustment elements of such article.

### DETAILED DESCRIPTION

The present disclosure relates to headgear lasing systems, and associated methods and apparel items, having utility for combating and reversing loss of head hair, and promoting hair growth on the head of a wearer of the headgear lasing system.

In one aspect, the present disclosure relates to a headgear lasing system, comprising a power supply that is configured to supply power to a laser for energizing the laser, wherein the laser when energized by the power supply emits laser radiation that is effective to combat hair loss. The laser in turn is coupled in laser radiation transmission relationship with a plurality of optical fibers so that the laser radiation is transmitted along the optical fibers to distal end portions thereof. The distal end portions of the optical fibers are secured to a support that is configured to overlie a head region, with distal ends of the optical fibers being arranged on the support to provide irradiation of such head region with the laser radiation.

The support may be in the form of a skull cap, or a mesh, framework, or scaffold structure that is configured to directly engage the head of a wearer, such as a skull cap that is formed of fabric, metal, polymeric material, wires, interlaced or spot-bonded strips, or other elements or materials that provide an appropriate support structure on which the distal end portions of the optical fibers can be mounted, to impinge lasing energy on the head of a wearer.

The support may itself be formed as an item of headgear apparel, e.g., as a cap or hat with a brim, a bill, earflaps, or other headgear features, or the support may be incorporated in a headgear article as a retrofitted or originally installed assembly therein. For example, the support may comprise a structure having a conformation of a spheroidal cap, with an inner concave face and an outer convex face, that is secured to an interior portion of a headgear article by any suitable securement elements, e.g., snap fasteners, hook and loop fasteners, button and button hole elements, zipper fasteners, buckle assemblies, clip structures, etc., wherein matably engageable securement elements are provided, on the support member and headgear article, respectively, so that the support is removably securable in the interior of the headgear article. Alternatively, the support may be sewn into, or otherwise permanently secured to the headgear article, e.g., by adhesive, riveting, melt fusion bonding, etc. As a still further alternative, the support may be constituted by a structural part of a headgear article, e.g., a crown portion of a cap, hat, bonnet, headscarf, bandanna, toboggan, helmet, etc.

The optical fibers at their distal end portions can be secured to the support in any suitable manner that enables transmission of the laser radiation conveyed by the fibers to the head of a wearer. The fiber distal end portions may for example be inserted and press-fit into grommets in the support so that the distal ends of the respective optical fibers face the scalp of the wearer, whereby lasing radiation is directed onto the scalp of the wearer of the headgear in which the support is present. In other embodiments, the fiber distal end portions may be clipped to the support, adhesively bonded to the support, mechanically fixtured to the support, or in other manner coupled or secured so that the optical fibers present their outlet faces to the scalp of the wearer and emit lasing radiation that is impinged on the scalp of the wearer. The support in other embodiments may comprise tubular guide members into which the optical fibers are introduced and secured, e.g., by crimping of the guide members to compressively hold the optical fibers in position.

The optical fibers are desirably arranged in an arrangement in which lasing radiation is transmitted from the fibers to a selected area of the scalp, e.g., an area of baldness on the scalp, so that the whole of such area is exposed to the lasing radiation. The end portions of the optical fibers for such purpose may be arranged in an array, such as a geometrically regular or irregular array, with center to center distance between faces of adjacent optical fibers in the array being such as to provide a desired uniformity of radiation dosage to the scalp.

In further embodiments, the headgear lasing system may further comprise one or more diffuser elements for diffusing the radiation emitted from the distal ends of the optical fibers over the scalp area to be exposed to the lasing radiation. The diffuser elements may comprise optically transparent or translucent material members that serve to distribute the lasing radiation over the area of the scalp to be treated, or may be of any other composition and structure that are efficacious to administer the lasing radiation to the head of the headgear wearer.

The laser radiation source may be of any suitable type that is effective to produce radiation that is safe and effective for promoting hair growth. The laser radiation source may for example comprise a low level radiation cool laser, such as a laser diode device that emits radiation that is effective to enhance head hair growth in relation to growth occurring in the absence of such radiation. The radiation may be of any suitable spectral regime. In some embodiments of the disclosure, the laser radiation source emits radiation at wavelength that is in a wavelength range of from 600 to 750 nm, e.g., a 650 nm laser radiation source. In other embodiments, the laser radiation source emits radiation at wavelength that is in a wavelength range of from 700 nm to 1 mm. In still other embodiments, the laser radiation source emits radiation at wavelength that is in a wavelength range of from 380 to 700 nm.

In one specific embodiment, the laser radiation source comprises a cool radiation laser diode that emits radiation at wavelength that is in a wavelength range of from 635 nm to 675 nm and an output laser power that is less than 5 milliwatts.

The headgear lasing system of the present disclosure may comprise a power supply that may be selectively manually actuated to energize the laser radiation source and initiate lasing radiation transmission through the optical fibers that are coupled to the laser radiation source, to the head of the wearer. The power supply may comprise a timer that is effective on actuation of the power supply to energize the laser radiation source only for a predetermined period of time, e.g., 15 or 20 minutes, or other period of time.

The headgear lasing system of the present disclosure in specific embodiments may comprise multiple laser radiation sources, each of which is coupled to multiple optical fibers arranged to deliver laser radiation to the head of the wearer. The use of multiple optical fibers with each laser radiation source permits small lasers and small power supplies to be employed.

In one implementation, the headgear lasing system of the disclosure is embodied in a baseball style cap having a skullcap support secured in the interior crown portion of the cap, with a patterned array of optical fibers fixtured in the support so that lasing radiation emitted from the end faces of the optical fibers is radially inwardly directed to the scalp of the cap wearer. The optical fibers are appropriately secured to the skullcap and extend to the laser radiation source device(s), being coupled to the laser radiation source device(s) so that laser radiation generated by the laser radiation source device(s) travels along the optical fibers and is emitted at distal end faces thereof, for impingement on the head of a wearer of the cap. The laser radiation source device(s) are coupled to a power supply that may be mounted for such purpose on an exterior surface of the cap, so as to be accessible for push-button actuation of the power supply to energize the laser radiation source device(s). As previously mentioned, the power supply may incorporate a timer mechanism that automatically shuts off after a predetermined period of time.

Thus, the present disclosure contemplates in various embodiments a headgear lasing system, comprising:

a power supply;
a laser device coupled in power receiving relationship to the power supply, and configured for generating laser radiation effective to promote growth of head hair;
a multiplicity of optical fibers coupled with the laser device to receive said laser radiation for transmission thereof through said optical fibers to respective end portions thereof; and
a support that is configured to overlie a head region, and to which the end portions of the optical fibers are secured so that the laser radiation transmitted through the optical fibers is directed therefrom to the head region.

The headgear lasing system may be constituted with the support being configured to engage the head of a wearer. The support may be configured to form a skull cap, mesh, framework, or scaffold structure, e.g., a skull cap that is formed of fabric, metal, polymeric material, wires, or interlaced or spot-bonded strips. The support may be configured to form a headgear article. The headgear lasing system may be constructed with the support secured, e.g., removably secured, to an interior portion of a headgear article. The support may be constituted by a structural part of a headgear article, e.g., a crown portion of a cap, hat, bonnet, headscarf, bandanna, toboggan, or helmet. The support in specific embodiments may comprise a structure having a conformation of a spheroidal cap, with an inner concave face and an outer convex face.

In the headgear lasing system, the end portions of the optical fibers may be arranged in an array, e.g., a geometrically regular array. The headgear lasing system in specific embodiments may further comprise a diffuser configured to diffuse radiation emitted from distal ends of the optical fibers, to distribute diffused radiation over a predetermined head region.

The laser device in the headgear lasing system may comprise a laser diode device or other suitable laser radiation source. In various embodiments, the laser device emits radiation at wavelength that is in a wavelength range of from 600 to 750 nm. In other embodiments, the laser device emits radiation at wavelength that is in a wavelength range of from 700 nm to 1 mm. In still other embodiments, the laser device emits radiation at wavelength that is in a wavelength range of from 380 to 700 nm. In additional embodiments, the laser device emits radiation at wavelength that is in a wavelength range of from 635 nm to 675 nm and produces an output laser power that is less than 5 milliwatts.

The headgear lasing system may be configured so that the power supply is manually actuable. In specific implementations, the power supply may comprise a timer control configured so that the power supply energizes the laser device for only a predetermined period of time, e.g., 15 to 20 minutes.

The disclosure contemplates embodiments in which the headgear lasing system comprises multiple ones of the laser device, each coupled with a separate multiplicity of optical fibers.

In one aspect, the disclosure contemplates headgear articles comprising the headgear lasing system as variously described herein. Such headgear article may for example comprise a cap, hat, bonnet, headscarf, bandanna, toboggan, or helmet. The headgear article may be constructed with the power supply mounted on an exterior surface of said article. In such headgear article, the support of the headgear lasing system may comprise an internal crown portion of such article.

In another aspect, the present disclosure relates to a method of enhancing growth of head hair of a subject in need thereof, comprising generating laser radiation effective for promoting growth of head hair, transmitting such radiation through a multiplicity of optical fibers, and impinging such radiation from distal end portions of the optical fibers on the head of the subject.

In such method, the distal end portions of the optical fibers can be mounted on or in a headgear article worn by the subject. The distal end portions of the optical fibers are advantageously arranged in an array, e.g., a geometrically regular array. The method may be conducted in various embodiments with the impinging radiation from distal end portions of the optical fibers being diffused prior to contact with the head of the subject. The laser radiation in the method of the present disclosure may be generated from a laser diode device, or other source of laser radiation of appropriate character. The laser radiation may be at wavelength that is in a wavelength range of from 600 to 750 nm, or at wavelength that is in a wavelength range of from 700 nm to 1 mm, or at wavelength that is in a wavelength range of from 380 to 700 nm. In one embodiment of the aforementioned methodology, the laser radiation is at wavelength that is in a wavelength range of from 635 nm to 675 nm and is produced at an output laser power that is less than 5 milliwatts. The radiation may be impinged on the head of said subject for a continuous period of 15 to 20 minutes, e.g., in a daily irradiation regimen.

Accordingly, the present disclosure contemplates a method of enhancing growth of head hair of a subject in need thereof, comprising disposing on the head of said subject a headgear lasing system of the present disclosure, and operating same to expose the head of the subject to said laser radiation.

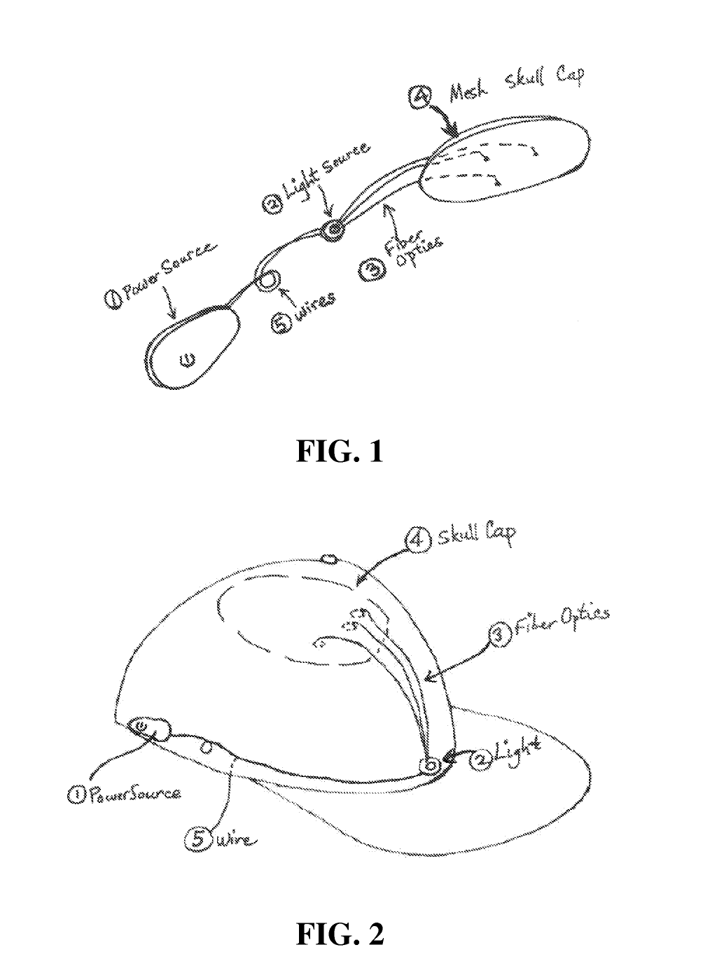

Referring now to the drawings, FIG. 1 is a schematic representation of a headgear lasing system according to one embodiment of the present disclosure. The headgear lasing system includes a power supply module 1, with a manually actuatable on-off push button. The power supply module 1 is coupled via electrical energy transmission wires 5 to a laser radiation source device 2. The laser radiation source device 2 is joined in light-transmission relationship to a multiplicity of optical fibers 3, with the optical fibers at their respective distal end portions being secured to a skullcap 4 constructed of a mesh fabric or other suitable material of construction.

The skullcap can be directly placed on the head, and the power supply module 1 can be actuated to energize the laser radiation source device 2 and generate laser radiation that is conveyed by the optical fibers 3 to the fiber end portions secured to the skullcap 4 so that lasing radiation is emitted from the end faces of the optical fibers and impinges the head of the wearer.

Alternatively, the skullcap can be secured to or otherwise incorporated in a headgear article. FIG. 2 is a schematic representation of a cap incorporating the headgear lasing system of FIG. 1, according to one embodiment of the present disclosure. In the FIG. 2 embodiment, the reference numbers of the headgear lasing system components are numbered correspondingly to the same components shown and described in reference to FIG. 1. It will be appreciated that the skullcap secured to the interior crown portion of the cap in FIG. 2 may be eliminated in favor of using the interior crown portion of the cap itself as the support for the optical fiber end portions.

The disclosure in another aspect relates to a scalp conditioning system including a laser source configured for generating laser radiation effective to promote growth of head hair, a vibration energy source configured for generating vibration energy, an array of bristle members arranged to overlie a scalp region so that terminal end portions of the bristle members are in contact with the scalp, such bristle members being optically coupled to the laser source and adapted to transmit lasing energy from the laser source to the scalp region when the laser source is energized, and such bristle members being coupled to the vibration energy source so that the terminal end portions of the bristle members provide vibratory massaging action on the scalp region, when the vibration energy source is energized.

The scalp conditioning system in various embodiments employs a laser source that emits radiation at wavelength that is in a wavelength range of from 600 to 750 nm. In other embodiments, the laser source emits radiation at wavelength that is in a wavelength range of from 700 nm to 1 mm. In other embodiments, the laser source employed in the scalp conditioning system emits radiation at wavelength that is in a wavelength range of from 382 700 nm. In still other embodiments, the laser source employed in the scalp conditioning system emits radiation at wavelength that is in a wavelength range of from 635 nm to 675 nm and produces an output laser power that is less than 5 mW.

The scalp conditioning system may further comprise a power supply that is actuatable to selectively energize the laser source and/or vibration energy source. The power supply may be manually actuatable in various embodiments. The power supply may comprise a timer control configured so that the power supply energizes the laser source and/or the vibration energy source for predetermined periods of time. For example, the power supply may comprise a timer control configured to energize the laser source for a period of 15 to 20 minutes.

The scalp conditioning system may utilize the array of bristle members as a structural assembly that can be reposed on the head of a subject, e.g., a human male or female subject afflicted with alopecia or other hair deficiency condition. Alternatively, the array of bristle members can be utilized as a structural assembly that is integrated in a headgear article, such as being mounted or positioned in the interior crown region of a hat, cap, scarf, bonnet, helmet, or other headgear member.

The bristle members of the scalp conditioning system may be configured for delivery of lasing energy to the scalp region in any suitable manner.

In some embodiments, the bristle members may be optical fibers, light pipes, waveguides, or the like that are transmissive of the lasing energy, so that the bristle members perform a direct lasing energy delivery function as well as functioning as vibratory elements for transmitting vibratory massaging energy to the scalp region. In such embodiments, the bristle members may be arranged in the array, coupled to the laser source and vibratory energy source in any suitable manner. For example, the bristle members at their proximal ends may be yoked together with one another in a bundle or other conformation, or such proximal ends may be connected to a central axle or collar member, or other unitary structure.

In other embodiments, the bristle members include hollow bristle elements having a bore therethrough, in which is disposed an optical fiber, light pipe, waveguide, or other optical transmission element, for conveying lasing energy to the distal end thereof for irradiation of the scalp region.

In still other embodiments, the bristle members may include bristle elements having coated or otherwise disposed thereon an optical fiber, light pipe, waveguide, or other optical transmission element, so that the optical fiber, light pipe, waveguide, or other optical transmission element longitudinally and circumferentially overlies the bristle element.

In still further embodiments, the vessel members may include bristle elements that are secured to optical transmission elements, e.g., wherein both of the bristle elements and optical transmission elements are of a fibrous or filament form, with each of such elements being spot-bonded or otherwise secured to the other. In a specific embodiment, the respective bristle and optical transmission elements may be disposed inside-by-side relationship to each other, and the contiguously associated bristle and optical transmission elements then may be disposed inside a shrink-wrap sleeve or other casing.

It will be recognized that the optical transmission and vibratory energy transmission functions may be carried out by bristle member constructions and arrangements that may be widely varied in the broad practice of the present disclosure, it being requisite only that the lasing energy and vibratory massaging energy be transmitted thereby to the scalp region of the subject when the scalp conditioning system is utilized to treat the scalp region of such subject.

The vibratory energy source utilized in the scalp conditioning system may be of any suitable type, and may for example comprise an electromechanical oscillator, vibrator, or other vibration energy generator.

The laser source and vibration energy source may be arranged in the scalp conditioning system to be concurrently operated. Alternatively, the system may be constituted so that each of the laser source and vibration energy source may be actuated separately so that only one of such sources, or alternatively both of such sources, can be selectively actuated. The system may therefore be constructed with an appropriate power source or supply that can be actuated to operate the laser source and/or vibration energy source as desired. In various embodiments, a power supply comprising one or more batteries are electrochemical cells is provided. In other embodiments, the power supply may include photovoltaic cells that are energized by ambient light, e.g., sunlight in an outdoor environment, or ultraviolet or other ambient light in an indoor setting. In specific embodiments, the scalp conditioning system may utilize a rechargeable battery that is chargeable with an associated inductive charging unit. It will be appreciated that the scalp conditioning system may be energized for laser energy impingement and/or massaging action, either concurrently or separately or sequentially, in any suitable manner and with any appropriate control and monitoring components. For example, the system may employ a controller incorporating a cycle timer so that the laser energy and massaging action are carried out for predetermined periods of time, or otherwise in a specified schedule and/or duration.

The scalp conditioning system in other embodiments may comprise a remote controller that is selectively actuatable to initiate or terminate the irradiation and/or massaging action of the system, e.g., by wireless radiofrequency communication, infrared signal communication, inductive switching, or other suitable remote control action.

When integrated in a headgear article, the scalp conditioning system may be disposed internally in the headgear or alternatively externally thereof, such as on an outer surface of a cap or hat, or it may be internally as well as externally disposed as regards its different component parts.

The scalp conditioning system as indicated may be configured in a suitable conformation for direct placement on the head of a wearer, or the scalp conditioning system may be integrated in a headgear article. When embodied in a headgear article, comprising a head engagement member, such as a cap, hat, scarf, helmet, toboggan, wig, headdress, bonnet, and bandanna, or the like, the head engagement member may be of an adjustable character to accommodate any of a variety of head sizes of the wearer. Such adjustability may be provided by an arrangement of coupling elements that may be selectively engaged at different dimensions to accommodate correspondingly variation in head size, such as snap fasteners, buttons, zippers, hook and loop fasteners, etc.

The disclosure correspondingly contemplates a scalp conditioning method in which lasing energy is impinged on a scalp region and vibratory massaging energy is transmitted to the scalp region, contemporaneously, sequentially, or otherwise selectively, from a scalp conditioning system of the disclosure, e.g., as deployed in a headgear article. In such manner, the lasing energy effective to promote growth of head hair is able to be transmitted to the scalp region contemporaneously with massaging action that is beneficial to promote blood circulation in the scalp region, as well as providing a soothing action that is conducive to the lasing energy treatment. Thus, one is incentivized by the massaging action to sustain a concurrent lasing energy impingement for a period of time that is useful to promote the hair growth action of the lasing energy treatment.

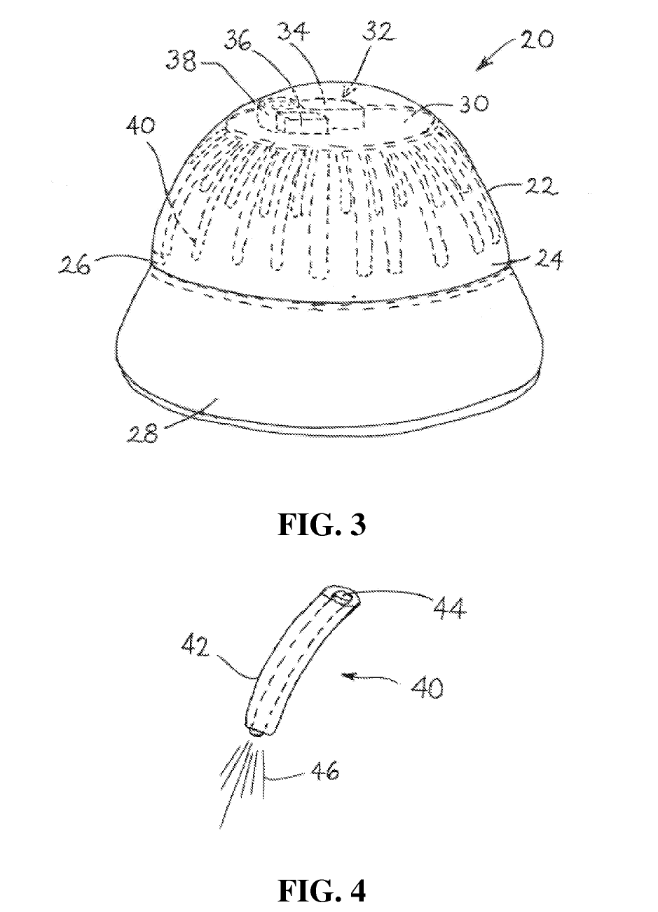

Accordingly, FIG. 3 is a schematic representation of a headgear article 20 according to another embodiment of the disclosure, providing capability of impinging lasing energy on the scalp of a wearer and/or massaging of the scalp of the wearer.

As illustrated in FIG. 3, the headgear article 20 comprises a cap 22 of head-mountable character, with an external surface 24 and an interior surface 26 bounding an interior volume of the cap, and a brim or bill 28. In the interior crown region of the cap 22 is secured a base member 30 that is affixed at its periphery to the interior surface 26 of the cap, such as by adhesive bonding, mechanical securement, a sewn seam, or the like. Mounted on the base member 30 is a scalp conditioning assembly 32 comprising a power supply 34, operatively coupled to a vibratory oscillator 36 and a laser source 38 so that the vibratory oscillator and laser source may be independently or conjointly selectively actuated. The vibratory oscillator and laser source are in turn operatively coupled to the bristle members 40.

The bristle members 40 are constituted to transmit lasing energy from the laser source 38 and vibratory massaging energy from the vibratory oscillator 36, to the scalp region of the wearer of the headgear article, with the distal end portions, e.g., end faces or distal tips, in contact with the scalp region of the wearer. By such arrangement, the laser source 38 and vibratory oscillator 36 can be actuated by the power supply 34, to transmit lasing energy effective for hair growth, and vibratory massaging energy, to the wearer's scalp region. For such purpose, the power supply may have an on-off switch that can be operated by the wearer to operate the lasing and massaging action, or selected ones thereof, or a remote control unit may be employed for such purpose.

Although the scalp conditioning assembly 32 is shown as being disposed in an interior compartment of the cap, it will be appreciated that such assembly can be mounted on an exterior surface 24 of the cap, such as on the crown of the cap, or on the brim or bill 28 thereof, with manual controls that can be readily accessed by the wearer of the headgear article.

FIG. 4 is a perspective view of a portion of a bristle member 40 of the headgear article of FIG. 3, incorporating an optical fiber 44 extending through a hollow bristle element 42 and emitting lasing energy 46 for impingement on the scalp in contact with or in proximity to the bristle member. The optical fiber at its proximal end is coupled to the laser source 38, four transmission of lasing energy through the fiber to the distal end for impingement on the scalp. The outer, circumscribing hollow bristle element 42 is coupled with the vibratory oscillator 36, so that the bristle member vibrates in response to action of the vibratory oscillator coupled therewith, to impart a vibratory massaging action on the scalp of the wearer contacting the bristle member.

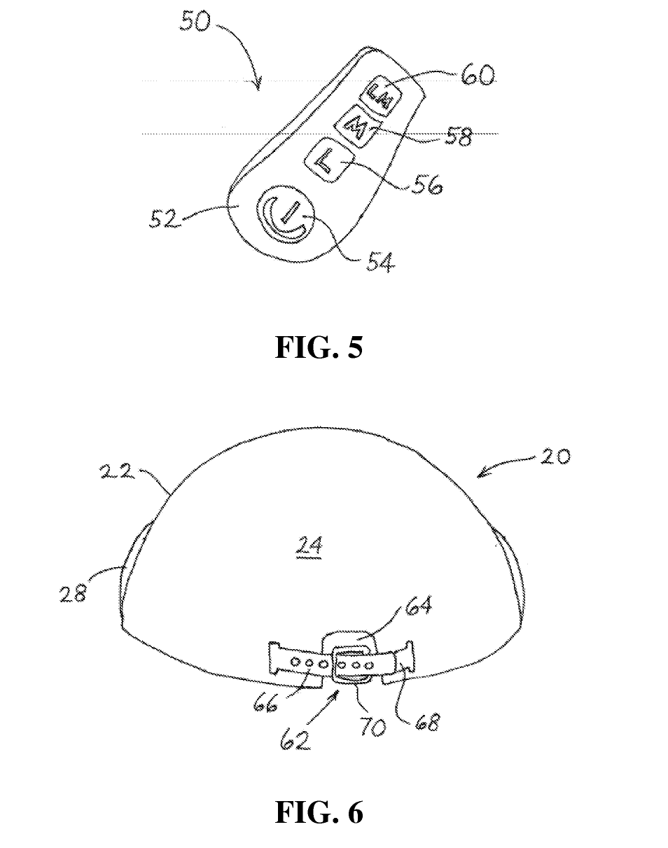

FIG. 5 is a perspective view of a remote control unit 50 that may be utilized with the headgear article shown in FIG. 3. In the illustrated embodiment, the remote control unit 50 comprises a housing 52 featuring an on-off switch 54, a manual button “L” for initiating operation of the laser source, a manual button “M” for initiating action of the vibratory oscillator, and a joint action manual button “LM” for initiating both operation of the laser source and action of the vibratory oscillator. The housing 52 contains appropriate circuitry coupled with the various switch 54 and button 56, 58, and 60 elements, for corresponding wireless control action of the scalp conditioning assembly 32 in the headgear article 20 shown in FIG. 3. The remote control unit 50 can operate by infrared signal generation, radiofrequency signal generation, or other signal generation modality, to provide for remote manual control of the lasing and massaging action of the scalp conditioning assembly.

FIG. 6 is a rear perspective view of the headgear article 20 of FIG. 3, showing the adjustment elements of such article in an adjustment assembly 62 thereof. The cap 22 is shown as having a rear expansion opening 64 on one side of which is arranged a tongue element 66 with openings therein engageable with the tang member of a buckle 70 mounted on strap 68, whereby the tongue element 66 may be translated to align one of the openings therein with the tang of the buckle 70 attached to strap 68, to effect adjustment of the cap to fit the head of a wearer.

The headgear article 20 shown in FIGS. 3-6 may be usefully employed to provide head hair growth-promoting treatment contemporaneously with scalp massage, as a therapeutically beneficial combination of treatment modalities. Such contemporaneous lasing/massage treatment may also be employed in conjunction with a treatment regimen of a topically applied hair growth formulation, such as the minoxidil-based topical composition disclosed in U.S. Pat. Nos. 7,749,489; 8,147,815; 8,119,111; and 8,444,960 of Shane Malek.

While the disclosure has been set out herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

What is claimed is:

1. A headgear lasing system, comprising:
a power supply;
a laser device coupled in power receiving relationship to the power supply, and configured for generating laser radiation effective to promote growth of head hair;
a multiplicity of optical fibers coupled with the laser device to receive said laser radiation for transmission thereof through said optical fibers to respective end portions thereof; and
a support that is configured to overlie a head region, and to which the end portions of the optical fibers are secured so that the laser radiation transmitted through the optical fibers is directed therefrom to the head region.

2. The headgear lasing system of claim 1, wherein the support is configured to form a headgear article, or to be secured to an interior portion of a headgear article, or wherein the support is constituted by a structural part of a headgear article.

3. The headgear lasing system of claim 1, wherein the end portions of the optical fibers are arranged in an array.

4. The headgear lasing system of claim 1, further comprising a diffuser configured to diffuse radiation emitted from distal ends of the optical fibers, to distribute diffused radiation over a predetermined head region.

5. The headgear lasing system of claim 1, wherein the laser device comprises a laser diode device.

6. The headgear lasing system of claim 1, wherein the laser device emits radiation at wavelength that is in a wavelength range selected from the group consisting of: a wavelength range of from 600 to 750 nm; a wavelength range of from 700 nm to 1 mm; a wavelength range of from 380 to 700; and a wavelength range of from 635 nm to 675 nm.

7. The headgear lasing system of claim 1, wherein the laser device produces an output laser power that is less than 5 milliwatts.

8. The headgear lasing system of claim 1, wherein the power supply is manually actuable, and comprises a timer control configured so that the power supply energizes the laser device for only a predetermined period of time.

9. The headgear lasing system of claim 1, comprising multiple ones of said laser device, each coupled with a separate multiplicity of optical fibers.

10. A headgear article comprising the headgear lasing system of claim 1.

11. A method of enhancing growth of head hair of a subject in need thereof, comprising generating laser radiation effective for promoting growth of head hair, transmitting said radiation through a multiplicity of optical fibers, and impinging said radiation from distal end portions of the optical fibers on the head of said subject.

12. The method of claim 11, wherein said distal end portions of the optical fibers are mounted on or in a headgear article worn by the subject.

13. The method of claim 11, wherein said radiation is impinged on the head of said subject for a predetermined continuous period in a daily irradiation regimen.

14. A method of enhancing growth of head hair of a subject in need thereof, comprising disposing on the head of said subject a headgear lasing system according to claim 1, and operating same to expose the head of said subject to said laser radiation.

15. A scalp conditioning system including a laser source configured for generating laser radiation effective to promote growth of head hair, a vibration energy source configured for generating vibration energy, and an array of bristle members arranged to overlie a scalp region so that terminal end portions of the bristle members are in contact with the scalp, such bristle members being optically coupled to the laser source and adapted to transmit lasing energy from the laser source to the scalp region when the laser source is energized, and such bristle members being coupled to the vibration energy source so that the terminal end portions of the bristle members provide vibratory massaging action on the scalp region, when the vibration energy source is energized.

16. The scalp conditioning system of claim 15, further comprising a power supply that is actuatable to selectively energize the laser source and/or vibration energy source.

17. The scalp conditioning system of claim 15, wherein the array of bristle members comprises bristle members that are transmissive of the lasing energy and vibratory massaging energy.

18. The scalp conditioning system of claim 15, wherein the vibration energy source comprises an electromechanical oscillator, vibrator, or vibration energy generator, and the scalp conditioning system further comprises a battery or photovoltaic cell power supply for energizing the laser source and/or vibration energy source.

19. A headgear article comprising the scalp conditioning system of claim 15.

20. A scalp conditioning method, comprising transmitting to a scalp region (i) lasing energy effective to promote hair growth, and (ii) vibratory massaging energy, from a scalp conditioning system according to claim 15, optionally wherein a topical hair growth formulation is administered to the scalp region prior to transmitting said lasing energy and vibratory massaging energy.
